(19) United States
(12) Patent Application Publication
(10) Pub. No.: US 2023/0201226 A1
(43) Pub. Date: Jun. 29, 2023

(54) COMBINATION THERAPY FOR REDUCING DRUG-INDUCED NEPHROTOXICITY, DYSLIPIDEMIA AND HYPERGLYCEMIA

(71) Applicant: Yissum Research Development Company of the Hebrew University of Jerusalem Ltd., Jerusalem (IL)

(72) Inventors: Yaakov NAHMIAS, Mevaseret Zion (IL); Aaron COHEN, Jerusalem (IL); Avner EHRLICH, Jerusalem (IL)

(73) Assignee: Yissum Research Development Company of the Hebrew University of Jerusalem Ltd., Jerusalem (IL)

(21) Appl. No.: 18/111,618

(22) Filed: Feb. 20, 2023

Related U.S. Application Data
(63) Continuation of application No. PCT/IL2021/051048, filed on Aug. 26, 2021.
(60) Provisional application No. 63/070,391, filed on Aug. 26, 2020.

(51) Int. Cl. A61K 31/665; A61K 31/352; A61K 31/122; A61K 38/13; A61P 13/12

(52) U.S. Cl. CPC A61K 31/665; A61K 31/352; A61K 31/122; A61K 38/13; A61P 13/12

(57) ABSTRACT

Methods, compositions and kits for reducing renal tissue toxicity in a subject caused by a kidney damaging agent are provided. The methods comprise administering to the subject: (i) a kidney damaging agent; (ii) a PPARA activator; and (iii) an inhibitor of a cellular pathway selected from the group consisting of C/EBP, PPARG, ER stress, GLUT2 and SGLT1/2; or (i) a kidney damaging agent; (ii) a SGLT2 inhibitor; and (iii) a PPARA activator, a C/EBP inhibitor, a PPARG inhibitor, or an ER stress inhibitor.

### RELATED APPLICATIONS

This application is a Continuation of PCT Patent Application No. PCT/IL2021/051048 having International filing date of Aug. 26, 2021, which claims the benefit of priority under 35 USC § 119(e) of U.S. Provisional Patent Application No. 63/070,391 filed on Aug. 26, 2020. The contents of the above applications are all incorporated by reference as if fully set forth herein in their entirety.

### FIELD AND BACKGROUND OF THE INVENTION

The present invention, in some embodiments thereof, relates to a combination therapy for reducing drug-induced nephrotoxicity, dyslipidemia and/or hyperglycemia by combining an agonist of PPARA and/or a GLUT-2 inhibitor with a suitable functional supplement.

Drug-induced nephrotoxicity is an extremely common condition and is responsible for a variety of pathological effects on the kidneys. It is defined as renal disease or dysfunction that arises as a direct or indirect result of exposure to drugs. The incidence of drug-induced nephrotoxicity has been increasing with the increasing use of prescription drugs and their easy availability as over-the-counter medications especially non-steroidal anti-inflammatory drugs (NSAIDs) or antibiotics. Drug-induced acute renal failure accounts for 20% of all acute renal failure cases. Among older adults, the incidence of drug-induced nephrotoxicity may be as high as 66%, due to a higher incidence of diabetes and cardiovascular diseases compelling them to take multiple medications. Although renal impairment is often reversible, it may still require multiple interventions and hospitalization. Most of the drugs which are found to be nephrotoxic exert toxic effects by one or more common pathogenic mechanisms. These include altered intraglomerular hemodynamics, tubular cell toxicity, inflammation, crystal nephropathy, rhabdomyolysis, and thrombotic microangiopathy. Knowledge of offending drugs and their particular pathogenic mechanisms of renal injury is critical for recognizing and preventing drug-induced renal impairment.

Cyclosporine A (CsA, also spelled as “Cyclosporin A”) is a very important immunosuppressive drug: it has been widely used in transplantation and greatly improves the survival rates of patients and grafts after solid-organ transplantation. However, the chronic use of CsA associates with high incidences of nephrotoxicity and the eventual development of chronic renal failure. Indeed, nephrotoxicity is the most frequent and clinically important complication of CsA use, especially in renal-transplant patients. CsA directly affects renal tubular epithelial cells; specifically, it promotes epithelial-mesenchymal transition, inhibits DNA synthesis and induces apoptosis. The CsA-induced apoptosis correlates with the oxidative stress, endoplasmic reticulum stress and autophagy that CsA causes. In humans and animals, the liver and intestines are the main sites where CsA is metabolized. The limit of intestinal metabolism causes the poor oral bioavailability of CsA in humans. CsA metabolites are generally less cytotoxic than the parent drug. However, higher concentrations of some CsA metabolites associate with nephrotoxicity in organ-transplant patients. Notably, compared to the liver, there is much less biotransformation of CsA in the kidney. This may explain why this drug is so nephrotoxic in vivo. In addition, the use of CsA for the treatment of rheumatoid arthritis can cause complications such as dyslipidemia and hyperglycemia.

Cisplatin, is a well-known chemotherapeutic drug. It has been used for treatment of numerous human cancers including bladder, head and neck, lung, ovarian, and testicular cancers. It is effective against various types of cancers, including carcinomas, germ cell tumors, lymphomas, and sarcomas. Its mode of action has been linked to its ability to crosslink with the purine bases on the DNA, interfering with DNA repair mechanisms, causing DNA damage, and subsequently inducing apoptosis in cancer cells. Dose-related and cumulative renal insufficiency, including acute renal failure, is the major dose-limiting toxicity of Cisplatin. Renal toxicity has been noted in 28% to 36% of patients treated with a single dose of 50 mg/m². It is first noted during the second week after a dose and is manifested by elevations in blood urea nitrogen (BUN) and creatinine, serum uric acid and/or a decrease in creatinine clearance. Renal toxicity becomes more prolonged and severe with repeated courses of the drug. Renal function must return to normal before another dose of Cisplatin can be given. Impairment of renal function has been associated with renal tubular damage. The administration of Cisplatin using a 6- to 8-hour infusion with intravenous hydration, and mannitol has been used to reduce nephrotoxicity. However, renal toxicity can still occur after utilization of these procedures. In addition, the use of the anti-cancer agent Cisplatin can cause complications such as dyslipidemia and hyperglycemia. Aminoglycoside antibiotics are widely used in the treatment of a variety of infections produced by Gram-negative bacteria and bacterial endocarditis. Their cationic structure, seems to have an important role in their toxicity, mostly affecting renal (nephrotoxicity) and hearing (ototoxicity) tissues in which they accumulate. In spite of their undesirable toxic effects, aminoglycoside antibiotics still constitute the only effective therapeutic alternative against germs insensitive to other antibiotics. This is primarily because of their chemical stability, fast bactericidal effect, synergy with beta lactam antibiotics, little resistance, and low cost. In spite of being one of the most nephrotoxic aminoglycoside antibiotic, gentamicin is still frequently used as a first- and second-choice drug in a vast variety of clinical situations. Moreover, gentamicin has been widely used as a model to study the nephrotoxicity of this family of drugs, both in experimental animals and human beings. In addition, the use of the anti-cancer agent Gentamicin can cause complications such as dyslipidemia and hyperglycemia.

Organ-on-a-chip applications allow the fabrication of minimal functional units of a single organ or multiple organs. Relevant to the field of nephrology, renal tubular cells have been integrated with microfluidic devices for making kidneys-on-a-chip. Although still early in development, kidneys-on-a-chip have shown potential to replace traditional animal and human studies. They either focus on the filtration unit (the glomerulus) or the tubular unit responsible for reabsorption and secretion. In the first type of kidney on a chip, human induced pluripotent stem cells (iPSC)-derived podocytes are combined with glomerular microvascular endothelial cells to make mature glomerular organoids (Hale, L. J., et al. Nat. Commun. 9, 5167 (2018) doi:10.1038/s41467-018-07594-z). Others developed organoids with multiple renal cell types from the glomerular and the tubular compartments but at the nephron progenitor cell level (Takasato, M., Nature 526, 564-568 (2015) doi:10.1038/nature15695; Jian Hui Low, Cell Stem Cell, Volume 25, Issue 3, 2019, Pages 373-387.e9, ISSN 1934-5909). The second type of kidney on a chip replicate the tubular system on a chip made of two chambers separated by a porous silicon membrane. The first compartment holds the proximal tubule cells, the second the endothelium (Kyung-Jin Jang, Integrative Biology, Volume 5, Issue 9, September 2013, Pages 1119-1129). In both systems, drug-induced nephrotoxicity can be studied at the end of the induction.

WO2013158143 teaches that SGLT-2 inhibitors can be used to reduce renal toxicity of glucose conjugated chemotherapeutic drugs such as glufosamide (also known as glucophosphamide).

Additional background art includes Lhotak et al., Am. J. Physiol. Renal. Physiol. 303: F266-F278, 2012.

### SUMMARY OF THE INVENTION

According to an aspect of some embodiments of the present invention there is provided a method for reducing renal tissue toxicity in a subject caused by a kidney-damaging agent, the method comprising administering to the subject:

(i) a kidney damaging agent;

(ii) a peroxisome proliferator-activated receptor alpha (PPARA) activator; and

(iii) an inhibitor of a cellular pathway selected from the group consisting of CCAAT/enhancer-binding protein (C/EBP), peroxisome proliferator-activated receptor gamma (PPARG), endoplasmic reticulum (ER) stress, glucose transporter 2 (GLUT2) and sodium-glucose cotransporter 1 and 2 (SGLT1/2).

According to an aspect of some embodiments of the present invention there is provided a method for reducing renal tissue toxicity in a subject caused by a kidney-damaging agent, the method comprising administering to the subject:

(i) a kidney damaging agent;

(ii) a sodium-glucose linked transporter type 2 (SGLT2) inhibitor; and

(iii) a peroxisome proliferator-activated receptor alpha (PPARA) activator, a CCAAT/enhancer-binding protein (C/EBP) inhibitor, a peroxisome proliferator-activated receptor gamma (PPARG) inhibitor, or an endoplasmic reticulum (ER) stress inhibitor.

According to an aspect to some embodiments of the invention there is provided a method for reducing renal tissue toxicity in a subject caused by a kidney-damaging agent, the method comprising administering to the subject:

(i) a kidney damaging agent;

(ii) a peroxisome proliferator-activated receptor alpha (PPARA) activator; and

(iii) a sodium-glucose linked transporter type 2 (SGLT2) inhibitor.

According to an aspect of some embodiments of the present invention there is provided an article of manufacture, comprising:

(i) a kidney damaging agent;

(ii) a peroxisome proliferator-activated receptor alpha (PPARA) activator; and

(iii) an inhibitor of a cellular pathway selected from the group consisting of CCAAT/enhancer-binding protein (C/EBP), peroxisome proliferator-activated receptor gamma (PPARG), endoplasmic reticulum (ER) stress, glucose transporter 2 (GLUT2) and sodium-glucose cotransporter 1 and 2 (SGLT1/2).

According to an aspect of some embodiments of the present invention there is provided an article of manufacture, comprising:

(i) a kidney damaging agent;

(ii) a sodium-glucose linked transporter type 2 (SGLT2) inhibitor; and

(iii) a peroxisome proliferator-activated receptor alpha (PPARA) activator, a CCAAT/enhancer-binding protein (C/EBP) inhibitor, a peroxisome proliferator-activated receptor gamma (PPARG) inhibitor, or an endoplasmic reticulum (ER) stress inhibitor.

According to an aspect of some embodiments of the present invention there is provided a composition comprising:

(i) a kidney damaging agent;

(ii) a PPARA activator; and

(iii) an inhibitor of a cellular pathway selected from the group consisting of C/EBP, PPARG, ER stress, GLUT2 and SGLT1/2.

According to an aspect of some embodiments of the present invention there is provided a composition comprising:

(i) a kidney damaging agent;

(ii) a SGLT2 inhibitor; and

(iii) a PPARA activator, a C/EBP inhibitor, a PPARG inhibitor, or an ER stress inhibitor.

According to some embodiments of the invention, the composition of some embodiments of the invention further comprising a pharmaceutically acceptable carrier.

According to some embodiments of the invention, at least the PPARA activator and the inhibitor in (iii) are in a co-formulation.

According to some embodiments of the invention, at least the SGLT2 inhibitor and the inhibitor in (iii) are in a co-formulation.

According to some embodiments of the invention, at least the SGLT2 inhibitor and the activator in (iii) are in a co-formulation.

According to some embodiments of the invention, the article of manufacture of some embodiments of the invention, for use in treating a disease for which the kidney damaging agent is therapeutic.

According to some embodiments of the invention, the kidney-damaging agent further causes dyslipidemia and/or hyperglycemia.

According to some embodiments of the invention, the inhibitor in (iii) is a naturally-occurring molecule.

According to some embodiments of the invention, the activator in (iii) is a naturally-occurring molecule.

According to some embodiments of the invention, the inhibitor in (iii) is a chemically-synthesized molecule.

According to some embodiments of the invention, the activator in (iii) is a chemically-synthesized molecule.

According to some embodiments of the invention, the method of some embodiments of the invention, the composition of some embodiments of the invention, or the article of manufacture of some embodiments of the invention, with the proviso that when the kidney damaging agent is glufosamide then the inhibitor is not a sodium-glucose transport protein 2 (SGLT2) inhibitor.

According to some embodiments of the invention, the method of some embodiments of the invention, the composition of some embodiments of the invention, or the article of manufacture of some embodiments of the invention, with the proviso that the kidney-damaging agent is not glufosamide.

According to some embodiments of the invention, the PPARA activator in (ii) is selected from the group consisting of Fenofibrate, Benzofibrate, Ciprofibrate, Gemfibrozil, and Clofibrate.

According to some embodiments of the invention, the SGLT2 inhibitor in (ii) is selected from the group consisting of: Empagliflozin, Dapagliflozin, Canagliflozin, Ertugliflozin, Ipragliflozin, Luseogliflozin, Remogliflozin etabonate, Sotagliflozin and Tofogliflozin.

According to some embodiments of the invention, the PPARA activator in (iii) is 9CLA.

According to some embodiments of the invention, the C/EBP inhibitor is Genistein.

According to some embodiments of the invention, the PPARG inhibitor is Luteolin.

According to some embodiments of the invention, the ER stress inhibitor is Quercetin.

According to some embodiments of the invention, the GLUT2 inhibitor is Phloretin.

According to some embodiments of the invention, the SGLT1/2 inhibitor is Phlorizin.

According to some embodiments of the invention, the subject has cancer and the kidney-damaging agent is a therapeutic agent used to treat the cancer.

According to some embodiments of the invention, the subject has undergone an organ or tissue transplant and the kidney-damaging agent is an immunosuppressive agent.

According to some embodiments of the invention, the subject has an infection and the kidney-damaging agent is used to treat the infection.

According to some embodiments of the invention, the kidney-damaging agent is a diagnostic agent.

According to some embodiments of the invention, the subject does not have a metabolic disease.

According to some embodiments of the invention, the subject does not have diabetes.

According to some embodiments of the invention, the kidney-damaging agent is selected from the group consisting of an NSAID, an ACE Inhibitor [Angiotensin-converting enzyme (ACE) inhibitor], an angiotensin II Receptor Blocker, an aminoglycoside antibiotic, a radiocontrast dye, cyclosporine A (CsA) and a chemotherapeutic agent.

According to some embodiments of the invention, the kidney-damaging agent is selected from the group consisting of cisplatin, gentamicin and Cyclosporine A.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E—Analysis of phospholipids and neutral lipids in human primary proximal tubule cells (hPTC) that were treated with either Cyclosporine A (1 μM) alone, or in combination with Empagliflozin (SGLT2i) at 5 μM or Fenofibrate at 20 μM, or in a further combination with Quercetin at 20 μM. Red=Phospholipidosis staining. Green=LipidTox stain to stain for neutral lipids. FIG. 1A—Cyclosporine A (1 μM) alone; FIG. 1B—Cyclosporine A (1 μM) and Fenofibrate at 20 μM; FIG. 1C—Cyclosporine A (1 μM) and Empagliflozin (SGLT2i) at 5 μM; FIG. 1D—Cyclosporin A (1 μM), Fenofibrate at 20 μM and Quercetin at 20 μM; FIG. 1E—Cyclosporin A (1 μM), Empagliflozin (SGLT2i) at 5 μM and Quercetin at 20 μM.

FIGS. 2A-E—Analysis of phospholipids and neutral lipids in human primary proximal tubule cells (hPTC) that were treated with either Cisplatin (1 μM) alone, or in combination with Empagliflozin (SGLT2i) at 5 μM or Fenofibrate at 20 μM, or in a further combination with Quercetin at 20 μM. Red=Phospholipidosis staining. Green=LipidTox stain to stain for neutral lipids. FIG. 2A—Cisplatin (1 μM) alone; FIG. 2B—Cisplatin (1 μM) and Fenofibrate at 20 μM; FIG. 2C—Cisplatin (1 μM) and Empagliflozin (SGLT2i) at 5 μM; FIG. 2D—Cisplatin (1 μM), Fenofibrate at 20 μM and Quercetin at 20 μM; FIG. 2E—Cisplatin (1 μM), Empagliflozin (SGLT2i) at 5 μM and Quercetin at 20 μM.

FIGS. 3A-E—Analysis of phospholipids and neutral lipids in human primary proximal tubule cells (hPTC) that were treated with either Gentamicin (1 mM) alone, or in combination with Empagliflozin (SGLT2i) at 5 μM or Fenofibrate at 20 μM, or in a further combination with Quercetin at 20 μM. Red=Phospholipidosis staining. Green=LipidTox stain to stain for neutral lipids. FIG. 3A—Gentamicin (1 mM) alone; FIG. 3B—Gentamicin (1 mM) and Fenofibrate at 20 μM; FIG. 3C—Gentamicin (1 mM) and Empagliflozin (SGLT2i) at 5 μM; FIG. 3D—Gentamicin (1 mM), Fenofibrate at 20 μM and Quercetin at 20 μM; FIG. 3E—Gentamicin (1 mM), Empagliflozin (SGLT2i) at 5 μM and Quercetin at 20 μM.

### DESCRIPTION OF SPECIFIC EMBODIMENTS OF THE INVENTION

The present invention, in some embodiments thereof, relates to a combination therapy for reducing drug-induced nephrotoxicity, dyslipidemia and/or hyperglycemia by combining an agonist of PPARA or a GLUT-2 inhibitor with a suitable functional supplement.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The kidney is an essential organ tasked with glucose and fluid homeostasis, as well as the excretion of drug metabolites. Drug-induced nephrotoxicity accounts for 20% of kidney failure in the general population, with incidence of drug-induced nephrotoxicity increasing to 66% in elderly patients taking prescription medication. The proximal tubule is particularly sensitive to drug toxicity due to its role in the concentration and reabsorption of metabolites. Importantly, clinically relevant drug-induced nephrotoxicity often occurs at plasma concentrations of the drug that are below the threshold of cellular damage in vitro. Thus, the mechanism of damage is unclear.

Cyclosporine A is given for treating rheumatoid arthritis where both dyslipidemia and hyperglycemia are major risk factors and complications.

Cisplatin and Gentamicin are anticancer drugs that cause hyperglycemia and dyslipidemia. This is a major complication of their use, and patients treated with Cisplatin or Gentamicin can be affected by diabetes as a side effect of the treatment.

The present inventors have uncovered that there are advantages to adding gliflozin and/or fibrate to the main drug (Cyclosporine A, Cisplatin or Gentamicin) beyond only reducing renal toxicity.

The present inventors uncovered that when using Cyclosporine A, e.g., for treating rheumatoid arthritis or cancer, the addition of gliflozin (an SGLT2 inhibitor) will reduce hyperglycemia and addition of fibrate (a PPARA agonist) would reduce dyslipidemia and inflammation.

Thus, the present inventors have devised a treatment regimen for treating a patient with a drug such a Cyclosporin, Cisplatin and Gentamicin, which may cause renal toxicity, with or without additional dyslipidemia and hyperglycemia. The treatment regimen comprises a combination therapy including for example an SGLT2 inhibitor (e.g., gliflozin) and a PPARA agonist (e.g., fibrate).

The present inventors have uncovered that in order to prevent renal toxicity, dyslipidemia and/or hyperglycemia in a patient treated with a drug such as Cyclosporine A, Cisplatin or Gentamicin, the patient should be further treated with a combination of a PPARA agonist and an inhibitor of a pathway selected from the group consisting of: C/EBP (e.g., Genistein), PPARG (e.g., Luteolin), ER stress (e.g., Quercetin), GLUT2 (e.g., Phloretin) and SGLT1/2 (e.g., Phlorizin). The use of such a combinational therapy prevents the renal toxicity, the dyslipidemia and/or the hyperglycemia associated with administration of the drug (e.g., Cyclosporine A, Cisplatin or Gentamicin) to the patient.

The present inventors have further uncovered that in order to prevent renal toxicity, dyslipidemia and/or hyperglycemia in a patient treated with a drug such as Cyclosporine A, Cisplatin or Gentamicin, the patient should be further treated with a combination of a SGLT2 inhibitor and an agent selected from the group consisting of a PPARA agonist (e.g., 9CLA), an inhibitor of C/EBP (e.g., Genistein), an inhibitor of PPARG (e.g., Luteolin), and an inhibitor of ER stress (e.g., Quercetin). The use of such a combinational therapy prevents the renal toxicity, the dyslipidemia and/or the hyperglycemia associated with administration of the drug (e.g., Cyclosporine, Cisplatin or Gentamicin) to the patient.

Table 1 below provides non-limiting exemplary PPARA agonists, and SGLT2 inhibitors which can be used in the combination therapy with a drug, such as Cyclosporine A, Cisplatin or Gentamicin, that can cause renal damage, dyslipidemia and/or hyperglycemia. Table 1 further provides a non-limiting list of exemplary functional supplements (e.g., naturally-occurring molecules) which can be added to the PPARA agonist and/or to the SGLT2 inhibitor when treating a subject with the drug which can cause renal damage, dyslipidemia and/or hyperglycemia.

TABLE 1
Table 1.

| PPARA Agonist | SGLT2 Inhibitors | |
|---|---|---|
| (Fibrates) | (Gliflozin) | Functional Supplement |
| Fenofibrate | Empagliflozin | 9CLA (PPARA agonist) |
| Bezafibrate | Dapagliflozin | Genistein (C/EBP |
| | | inhibitor) |
| Ciprofibrate | Canagliflozin | Luteolin (PPARG |
| | | inhibitor) |
| Gemfibrozil | Ertugliflozin | Quercetin (ER stress |
| | | inhibitor) |
| Clofibrate | Ipragliflozin | Phloretin (GLUT2 |
| | | inhibitor) |
| | Luseogliflozin | Phlorizin (SGLT1/2 |
| | | inhibitor) |
| | Remogliflozin etabonate | |
| | Sotagliflozin | |
| | Tofogliflozin | |

According to some embodiments of the invention the combination therapy of an SGLT2 inhibitor and an ER stress inhibitor in renal cells, which are subjected to (e.g., exposed, being in contact), a kidney damaging agent, reduces by at least 20% (e.g., by at least 30%, 40%, 50%, 60%, 70%, 80%, 90% or more) the level of phospholipids in the renal cells as compared to the level of phospholipids in renal cells, which are subjected to a kidney damaging agent, and which are treated with only an SGLT2 inhibitor.

According to some embodiments of the invention the combination therapy of an SGLT2 inhibitor and an ER stress inhibitor in renal cells, which are subjected to a kidney damaging agent, reduces by at least 20% (e.g., by at least 30%, 40%, 50%, 60%, 70%, 80%, 90% or more) the level of phospholipids and neutral lipids in the renal cells as compared to the level of phospholipids and neutral lipids in renal cells, which are subjected to a kidney damaging agent, and which are treated with only an SGLT2 inhibitor.

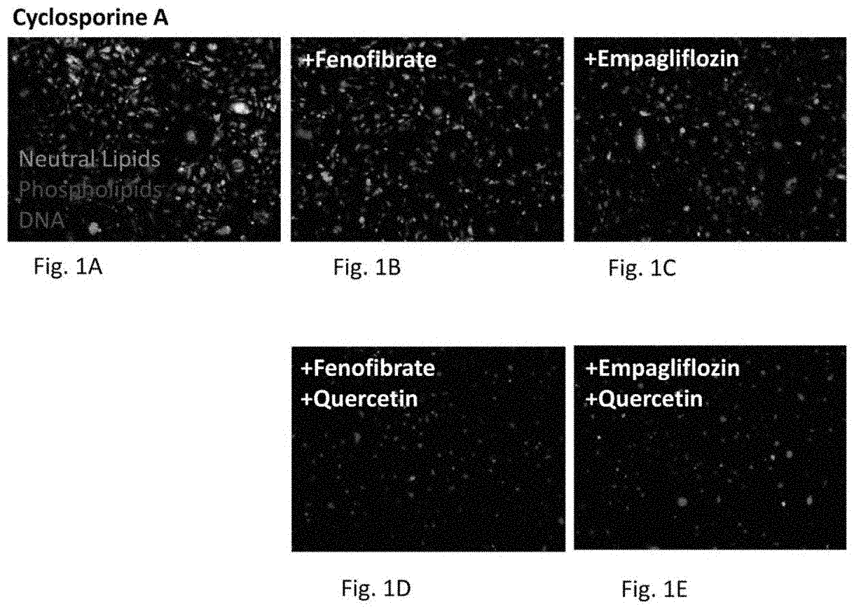

Example 1 of the Examples section which follows shows that a combination of an SGLT2 inhibitor (e.g., gliflozin such as Empagliflozin) together with an ER stress inhibitor such as Quercetin reduces both phospholipids and neutral lipids (FIGS. 1A-C, 2A-C and 3A-C) in renal cells that were treated with Cyclosporin A, Cisplatin and Gentamicin.

According to some embodiments of the invention the combination therapy of a PPARA agonist and an ER stress inhibitor in renal cells, which are subjected to a kidney damaging agent, reduces by at least 20% (e.g., by at least 30%, 40%, 50%, 60%, 70%, 80%, 90% or more) the level of phospholipids in the renal cells as compared to the level of phospholipids in renal cells, which are subjected to a kidney damaging agent and which are treated with only a PPARA agonist.

According to some embodiments of the invention the combination therapy of a PPARA agonist and an ER stress inhibitor in renal cells, which are subjected to a kidney damaging agent, reduces by at least 20% (e.g., by at least 30%, 40%, 50%, 60%, 70%, 80%, 90% or more) the level of phospholipids and neutral lipids in the renal cells as compared to the level of phospholipids and neutral lipids in renal cells, which are subjected to a kidney damaging agent and which are treated with only a PPARA agonist.

Example 1 of the Examples section which follows shows that a combination of a PPARA agonist (e.g., fibrate, such as Fenofibrate) together with an ER stress inhibitor such as Quercetin reduces both phospholipids and neutral lipids (FIGS. 1A-C, 2A-C and 3A-C) in renal cells that were treated with Cyclosporin A, Cisplatin and Gentamicin.

According to an aspect to some embodiments of the invention there is provided a method of reducing renal tissue toxicity in a subject caused by a kidney-damaging agent, the method comprising administering to the subject:

(i) a kidney damaging agent;

(ii) a peroxisome proliferator-activated receptor alpha (PPARA) activator; and

(iii) an inhibitor of a cellular pathway selected from the group consisting of CCAAT/enhancer-binding protein (C/EBP), peroxisome proliferator-activated receptor gamma (PPARG), endoplasmic reticulum (ER) stress, glucose transporter 2 (GLUT2) and sodium-glucose cotransporter 1 and 2 (SGLT1/2).

According to an aspect to some embodiments of the invention there is provided a method for reducing renal tissue toxicity in a subject caused by a kidney damaging agent, the method comprising administering to the subject:

(i) a kidney damaging agent;

(ii) a sodium-glucose linked transporter type 2 (SGLT2) inhibitor; and

(iii) a peroxisome proliferator-activated receptor alpha (PPARA) activator, a CCAAT/enhancer-binding protein (C/EBP) inhibitor, a peroxisome proliferator-activated receptor gamma (PPARG) inhibitor, or an endoplasmic reticulum (ER) stress inhibitor.

According to an aspect to some embodiments of the invention there is provided a method for reducing renal tissue toxicity in a subject caused by a kidney-damaging agent, the method comprising administering to the subject:

(i) a kidney damaging agent;

(ii) a peroxisome proliferator-activated receptor alpha (PPARA) activator; and

(iii) a sodium-glucose linked transporter type 2 (SGLT2) inhibitor.

As used herein, the term “kidney damaging agent” refers to a therapeutic agent, a diagnostic agent, an imaging agent (e.g. dyes), a food, a cosmetic, which when used at clinically acceptable doses causes unwanted damage to the kidney.

The damage to the kidney can be a disruption of vital cellular function such as macromolecule homeostasis (e.g., lipid accumulation, and/or abnormal formation of lipid structure), micromolecule and ion transport (e.g., glucose secretion and/or absorption, sodium transport, and/or water transport), ultrastructure and cellular organization (e.g., loss of polarity, hypertrophy, and/or formation of new vesicles).

According to some embodiments of the invention, the kidney damaging agent typically has a primary function that brings about a diagnostic or a therapeutic effect and has a negative side-effect of causing damage to renal tissue.

In one embodiment, the kidney damaging agent is one that disrupts the polarity of the proximal tubule epithelial cells.

The term “renal tissue” refers to any tissue of the kidney. In one embodiment the renal tissue comprises renal tubules, especially proximal tubule cells.

As used herein, the term “subject” includes mammals, preferably human beings at any age.

According to some embodiments of the invention, the subject is exposed to or treated with the kidney damaging agent.

According to some embodiments of the invention, the subject may suffer from a pathology or a condition which requires treatment with a kidney damaging agent.

According to some embodiments of the invention, the subject may be in need of a diagnostic procedure employing a kidney damaging agent, e.g., for enhancing imaging.

According to some embodiments of the invention, the subject has cancer and the kidney damaging agent is a therapeutic agent used to treat the cancer.

According to some embodiments of the invention, the subject has undergone an organ or tissue transplant and the kidney damaging agent is an immunosuppressive agent.

According to some embodiments of the invention, the subject has an infection and the kidney damaging agent is used to treat the infection.

According to some embodiments of the invention, the kidney damaging agent is a diagnostic agent.

According to some embodiments of the invention, the subject does not have a metabolic disease.

According to some embodiments of the invention, the subject does not have diabetes.

Non-limiting examples of therapeutic agents which are known to damage the kidney are provided in Table 2.

TABLE 2
Table 2.

| Medication | Drug category | Renal toxicity |
|---|---|---|
| Acetaminophen | Non-narcotic | Chronic interstitial |
| | analgesic | nephritis, acute tubular |
| | | necrosis |
| Acetazolamide | Carbonic-anhydrase | Proximal renal tubular |
| | inhibitor | acidosis |
| Acyclovir | Antiviral | Acute interstitial |
| | | nephritis, crystal |
| | | nephropathy |
| Allopurinol | Hypouricemic agent | Acute interstitial |
| | | nephritis |
| Aspirin | Non-narcotic | Chronic interstitial |
| | analgesic | nephritis |
| Amitriptyline | Antidepressant | Rhabdomyolysis |
| Aminoglycosides | Antimicrobial | Acute tubular necrosis |
| Amphotericin B | Antifungal | Acute tubular necrosis, |
| | | distal renal tubular |
| | | acidosis |
| Angiotensin- | Antihypertensive | Acute kidney injury |
| converting enzyme | | |
| inhibitors (ACEI) | | |
| Angiotensin | Antihypertensive | Acute kidney injury |
| receptor | | |
| blockers (ARB) | | |
| Benzodiazepines | Sedative-Hypotonic | Rhabdomyolysis |
| Beta lactams | Antimicrobial | Acute interstitial |
| | | nephritis |
| Carbenicillin | Antimicrobial | Metabolic alkalosis |
| Cephalosporin | Antimicrobial | Acute tubular necrosis |
| Cholpropamide | Sulfonylureas | Hyponatremia, syndrome |
| | | inappropriate ADH |
| | | secretion |
| Cimetidine | Gastrointestinal | Acute interstitial |
| | | nephritis |
| Cisplatin | Antineoplastic | Chronic interstitial |
| | | nephritis |
| Clopidogrel | Antiplatelet | Thrombotic miroangiopathy |
| Cocaine | Narcotic | Rhabdomyolysis |
| | analgesic | |
| Contrast agents | Contrast medium | Acute tubular necrosis |
| Cortisone | Corticosteroid | Metabolic alkalosis, |
| | | hypertension |
| Cyclophosphamide | Antineoplastic | Hemorrhagic cystitis |
| Cyclosporine | Immunosuppressive | Acute tubular necrosis, |
| | | chronic interstitial |
| | | nephritis, thrombotic |
| | | microangiopathy |
| D-penicillamine | Antirheumatic | Nephrotic syndrome |
| Diphenhydramine | Antihistamine | Rhabdomyolysis |
| Furosemide | Loop diuretic | Acute interstitial nephritis |
| Ganciclovir | Antiviral | Crystal nephropathy |
| Gold Na thiomalate | Aniarthritic | Glomerulonephritis, |
| | | nephrotic syndrome |
| Haloperidol | Antipsychotic | Rhabdomyolysis |
| Indinavir | Antiviral | Acute interstitial |
| | | nephritis, crystal |
| | | nephropathy |
| Interferon-alfa | Antineoplastic | Glomerulonephritis |
| Lansoprazole | Proton pump | Acute interstitial |
| | inhibitor | nephritis |
| Lithium | Antipsychotic | Chronic interstitial |
| | | nephritis, |
| | | glomerulonephritis, |
| | | rhabdomyolysis |
| Methadone | Narcotic | Rhabdomyolysis |
| | analgesic | |
| Methamphetamine | Psychostimulant | Rhabdomyolysis |
| Methotrexate | Antineoplastic | Crystal nephropathy |
| Mitomycin-C | Antineoplastic | Thrombotic |
| | | microangiopathy |
| Naproxen | Nonsteroidal anti- | Acute and chronic |
| | inflammatory | interstitial nephritis, |
| | | acute tubular necrosis, |
| | | glomerulonephritis |
| Omeprazole | Proton pump | Acute interstitial |
| | inhibitor | nephritis |
| Pamidronate acid | Bisphosphonate, | Glomerulonephritis |
| | osteoporosis | |
| | prevention | |
| Pantoprazole | Proton pump | Acute interstitial |
| | inhibitor | nephritis |
| Penicillin G | penicillin | Glomerulonephritis |
| Pentamidine | Antimicrobial | Acute tubular necrosis |
| Phenformin | Hypoglycemic | Lactic acidosis |
| Phenacetin | Non-narcotic | Chronic interstitial |
| | analgesic | nephritis |
| Phenytoin | Anticonvulsant | Acute interstitial |
| | | nephritis, diabetes |
| | | insipidus |
| Probenecid | Uricosuric | Crystal nephropathy, |
| | | nephrotic syndrome |
| Puromycin | Antimicrobial | Nephrotic syndrome |
| Quinine | Muscle relaxant | Thrombotic |
| | | microangiopathic |
| Quinolones | Antimicrobial | Acute interstitial |
| | | nephritis, crystal |
| | | nephropathy |
| Rifampin | Antimicrobial | Acute interstitial |
| | | nephritis |
| Ranitidine | Gastrointestinal | Acute interstitial |
| | | nephritis |
| Statins | Lipid- lowering | Rhabdomyolysis |
| Sulfonamides | Antimicrobial | Acute interstitial |
| | | nephritis, crystal |
| | | nephropathy |
| Tacrolimus | Immunosuppressive | Acute tubular necrosis |
| Tetracycline | Antimicrobial | Acute tubular necrosis |
| azides | Diuretic | Acute interstitial |
| | | nephritis |
| Tolbutamide | Hypoglycemic | Nephrotic syndrome |
| Vancomycine | Antimicrobial | Acute interstitial |
| | | nephritis |

According to some embodiments of the invention, the kidney damaging agent is selected from the group consisting of an NSAID, an ACE Inhibitor, an angiotensin II Receptor Blocker, an aminoglycoside antibiotic, a radiocontrast dye, cyclosporine A (CsA) and a chemotherapeutic agent.

According to some embodiments of the invention, the kidney damaging agent is a contrast agent, such as a radiocontrast dye.

According to some embodiments of the invention, the kidney damaging agent is selected from the group consisting of cisplatin, gentamicin and Cyclosporine A.

According to some embodiments of the invention, the kidney damaging therapeutic agent is gentamycin.

According to some embodiments of the invention, the kidney damaging therapeutic agent is cyclosporine A.

According to some embodiments of the invention, the kidney damaging therapeutic agent is cisplatin.

In the case when the kidney damaging agent is a therapeutic agent, the kidney damaging agent is provided to the subject in order to treat the disease. In one embodiment, the subject is in chronic need of the therapeutic agent.

According to a specific embodiment, the subject does not have a metabolic disease including for example diabetes, atherosclerosis and lipid disorders including primary elevated cholesterol, dyslipidemic syndrome, primary elevated triglycerides, primary low-HDL syndromes, Familial hypercholesterolemia, (a genetic disorder that increases total and LDL cholesterol) and familial hypertriglyceridemia.

Preferably, the kidney damaging agent is not being used to treat an underlying kidney disease.

Thus, for example in the case of cisplatin, the subject of this aspect of the present invention has a cancer (e.g. testicular cancer, ovarian cancer, cervical cancer, breast cancer, bladder cancer, head and neck cancer, esophageal cancer, lung cancer, mesothelioma, brain tumors or neuroblastoma). In the case of cyclosporine A, the subject of this aspect of the present invention may have a disease such as rheumatoid arthritis, psoriasis. Crohn's disease, or may have had an organ transplant. In the case of gentamycin, the subject of this aspect of the present invention may have a bacterial infection, including but not limited to include bone infections, endocarditis, pelvic inflammatory disease, meningitis, pneumonia, urinary tract infections, and sepsis.

According to some embodiments of the invention, the kidney damaging agent causes cellular dyslipidemia and/or hyperglycemia.

As used herein the term “dyslipidemia” refers to a condition characterized by having abnormal content (above or below), or distribution of cellular lipids such as triglyceride(s), phospholipid(s), free fatty acid(s) and cholesterol as compared to the levels or distribution pattern in a healthy subject.

In some embodiments, the kidney damaging agent causes steatosis (e.g., a condition caused by high tissue lipid levels).

Dyslipidemia can be detected using various imaging methods such as magnetic resonance imaging (MRI) and/or ultrasound.

Additionally or alternatively, dyslipidemia can be detected in vitro using a biological sample of the subject. For example, a biological sample comprising kidney cells of a subject can be a urine sample (which can be collected in a non-invasive way), or a tissue biopsy of the subject's kidney (e.g., obtained by a fine needle aspiration or using a surgical tool).

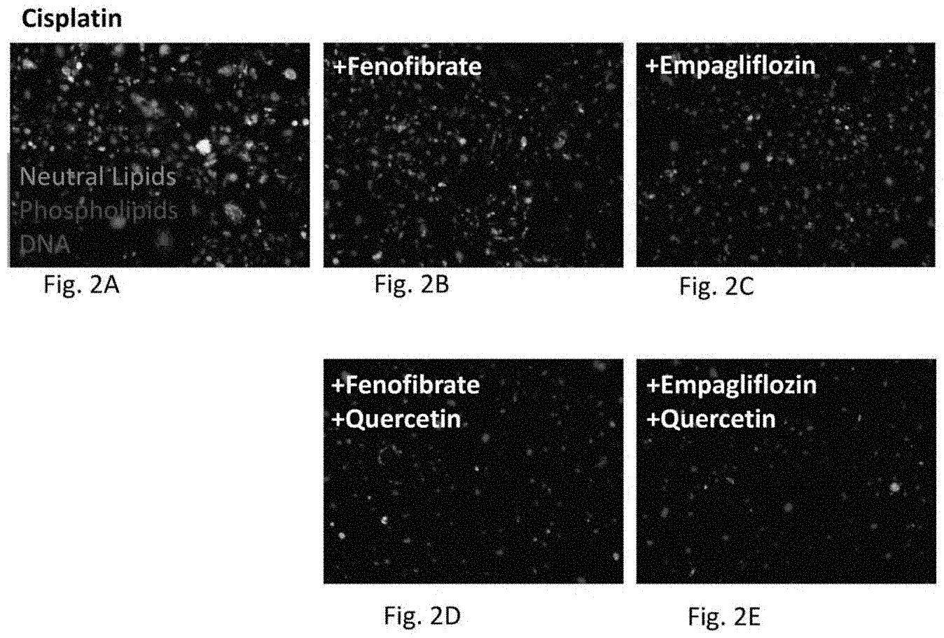

Once obtained, the kidney cells can be evaluated at the cellular level for the presence, absence and/or degree of steatosis. For example, steatosis can be evaluated at a cellular level, e.g., by detecting the level of phospholipids (e.g., using the Red Phospholipidosis staining reagent) and/or the level of neutral lipids (e.g., using the LipidTox stain). This is demonstrated in FIGS. 1A, 2A and 3A in response to treatment of cells with cyclosporine A, Cisplatin and Gentamicin, respectively.

As used herein the term “hyperglycemia” refers to a condition characterized by high tissue glucose levels.

Hyperglycemia can be detected using imaging techniques such as positron emission tomography (PET) scan, in which a labeled glucose molecule (e.g., radioactively-labeled glucose) is injected into the subject and the accumulation of glucose in the tissue is detected.

Additionally or alternatively, hyperglycemia can be detected in vitro using a biological sample of the subject, such as from a urine sample or a tissue biopsy of the kidney.

Once obtained, the kidney cells can be evaluated at the cellular level for the presence, absence and/or degree of hyperglycemia. For example, hyperglycemia can be evaluated at the cellular level, e.g., by the increase in the glucose retention as detected by 2-NBDG assay (2-(N-(7-Nitrobenz-2-oxa-1,3-diazol-4-yl)Amino)-2-Deoxyglucose).

As used herein the term “activator” refers to a molecule which directly (i.e., by direct interaction with the indicated target) or indirectly activates a signaling pathway.

As used herein the term “inhibitor” refers to a molecule which inhibits a component of a signaling pathway, or an activator of effector of the component in the signaling pathway.

According to some embodiments of the invention, the inhibitor is a naturally-occurring molecule.

As used herein the phrase “naturally-occurring molecule” refers to a molecule found in nature and has not undergone a synthetic chemical manipulation.

It should be noted that the naturally-occurring molecule can be available with a suitable carrier, e.g., an ethanol extract, a water-based medium, and the like, which maintains the function of the naturally-occurring molecule.

It is also noted that the naturally-occurring molecule can be used as food supplement or as a prescription drug which may include a label of notice for approval for use by the U.S. Food and Drug Administration.

According to some embodiments of the invention, the inhibitor is a chemically synthesized molecule.

As described, the method according to some embodiments of the invention requires a combination of protective agents for reducing the renal toxicity that may be caused by the kidney damaging agent. The protective agents comprise:

a peroxisome proliferator-activated receptor alpha (PPARA) activator; and

an inhibitor of a cellular pathway selected from the group consisting of CCAAT/enhancer-binding protein (C/EBP), peroxisome proliferator-activated receptor gamma (PPARG), endoplasmic reticulum (ER) stress, glucose transporter 2 (GLUT2) and sodium-glucose cotransporter 1 and 2 (SGLT1/2).

The peroxisome proliferator-activated receptor alpha (PPARA) is a nuclear transcription factor belonging to the steroid hormone receptor superfamily. Peroxisome proliferators include hypolipidemic drugs, herbicides, leukotriene antagonists, and plasticizers. PPARs affect the expression of target genes involved in cell proliferation, cell differentiation and in immune and inflammation responses.

A PPARA activator such a fibrate induces nuclear localization of PPARα.

Non-limiting examples of a PPARA activators include fibrate drugs, a class of amphipathic carboxylic acids such as clofibrate, gemfibrozil, ciprofibrate, bezafibrate, and fenofibrate.

Additional examples of PPARA activators, include, but are not limited to, naturally-occurring PPARA activators such as natural fatty acids (such as linoleic, monotriajaponide A), conjugated fatty acids (such as Conjugated (9Z,11E)-Linoleic acid), Omega 3 polyunsaturated fatty acids (such as Hexadecatrienoic acid, Alpha-linolenic acid, Stearidonic acid, Eicosatrienoic acid, Eicosatetraenoic acid, Eicosapentaenoic acid, Heneicosapentaenoic acid, Docosapentaenoic acid, Docosahexaenoic acid, Tetracosapentaenoic acid, Tetracosahexaenoic acid), Terpenes (such as linalool, trans-Caryophyllene, farnesol, Phytol, oleanolic acid and Fucoxanthin), Polyketides (such as monotriajaponide A, bilobetin and norathyriol), Phenylpropanoids (such as Rosmarinic acid, coumarin umbelliferone, Sesamin), Polyphenols (such as naringenin, resveratrol and bilobetin), and Alkaloids (such as picrasidine C, berberine and oxymatrine).

Additional examples of PPARA activators, include, but are not limited to, chemically-synthesized PPARA activators such as synthetic fatty acids such as (2-(4-(2-(1-Cyclohexanebutyl)-3-cyclohexylureido)ethyl)phenylthio)-2-methylpropionic acid; GW7647, KDS-5104; N-[(1R)-2-hydroxy-1-methylethyl-9Z-octadecenamide; AM3102, (S)-2-(3-(1-(2-(4-isopropylphenyl)acetyl)piperidin-3-yl)phenoxy)-2-methylpropanoic acid; CP 775146, 2-[[4-[2-[[[(2, 4-difluorophenyl)amino]carbonyl]heptylamino]ethyl]phenyl]thio]-2-methyl-propanoic acid; GW 9578, 2-Methyl-2-[4-[[[[4-methyl-2-[4-(trifluoromethyl)phenyl]-5-thiazolyl]carbonyl]amino]methyl]phenoxy]propanoic Acid; GW 590735), and Sythethic pyrimidines (such as Pirinixic acid; WY 14643).

According to some embodiments of the invention, the PPARA activator is selected from the group consisting of Fenofibrate, Benzofibrate, Ciprofibrate, Gemfibrozil, and Clofibrate.

According to some embodiments of the invention, the inhibitor of a cellular pathway selected from the group consisting of C/EBP, PPARG, ER stress, GLUT2 and SGLT1/2 is a naturally-occurring molecule.

According to some embodiments of the invention, the inhibitor of a cellular pathway selected from the group consisting of C/EBP, PPARG, ER stress, GLUT2 and SGLT1/2 is a chemically synthesized molecule.

As used herein, the term “glucose transporter” refers to a protein that transports compounds (whether glucose, glucose analogs, other sugars such as fructose or inositol, or non-sugars such as ascorbic acids) across a cell membrane and are members of the glucose transporter “family” based on structural similarity (e.g., homology to other glucose transport proteins). Glucose transporters also include transporter proteins that have a primary sugar substrate other than glucose. For example, the glucose transporter GLUTS is primarily a transporter of fructose, and is reported to transport glucose itself with low affinity. Similarly, the primary substrate for the glucose transporter HMIT is myo-inositol (a sugar alcohol). Examples of glucose transporter include, but are not limited to GLUT1-12, HMIT and SGLT1-6 transporters.

Non-limiting examples of GLUT2 inhibitors which are naturally-occuring include a flavonoid, such as a flavonol, such as a quercetin selected from the group consisting of: Phloretin, aglycone quercetin, quercetin glycoside, isoquercetin, Quercetin 3-O-β-glucuronide, cyanidin 3-glucoside, cyanin, Spriaeoside, Fisetin, Myricetin; a Flavone (such as Apigenin, and Luteolin), and a Flavanone (e.g., Hesperetin).

A non-limiting example of a naturally-occuring SGLT1/2 inhibitor is Phlorizin.

Non-limiting examples of chemically-synthesized SGLT1/2 inhibitors include Dapagliflozin (an SGLT2 inhibitor specific to the kidneys), canagliflozin (Invokana, Janssen pharmaceuticals), dapagliflozin (Forxiga [known as Farxiga in the USA]), empagliflozin (Jardiance), and ertugliflozin.

Other contemplated GLUT2 transporter naturally occurring inhibitors include: sappanin-type (SAP) homoisoflavonoids (PMID: 29533635), quercetin, myricetin, isoquercitrin (PMID: 17172639), Phloretin, and Phlorizin (dual inhibitor).

According to some embodiments of the invention, with the proviso that when the kidney damaging agent is glufosamide then the inhibitor is not as sodium-glucose transport protein 2 (SGLT2) inhibitor.

According to some embodiments of the invention, with the proviso that the kidney damaging agent is not glufosamide.

As used herein the term “peroxisome proliferator-activated receptor gamma (PPARG)” refers to a nuclear receptor which is a regulator of adipocyte differentiation, and has been implicated in the pathology of numerous diseases including obesity, diabetes, atherosclerosis and cancer.

According to some embodiments of the invention, the PPARG inhibitor is a naturally-occurring molecule selected from the group consisting of luteolin, Quercetin, rosmarinic acid, 7-chloroarctinone-b, protopanaxatriol, tanshinone HA, astaxanthin, foenumoside B and diosmetin.

As used herein the term “CCAAT/enhancer-binding protein (C/EBP)” refers to a transcription factor that contains a basic leucine zipper (bZIP) domain and recognizes the CCAAT motif in the promoters of target genes. C/EBP alpha functions in homodimers and also heterodimers with C/EBP beta and gamma.

According to some embodiments of the invention, the C/EBP inhibitor is a naturally-occurring molecule selected from the group consisting of Genistein, Betulinic Acid, Celastrol, and Withaferin A.

As used herein the phrase “endoplasmic reticulum (ER) stress” refers to multiple stress response pathways such as IRE1 (endoplasmic reticulum to nucleus signaling 1, also nkown as ERN1), PERK (eukaryotic translation initiation factor 2 alpha kinase 3, also known as EIF2AK3), ATF6 (activating transcription factor 6; also known as ACHM7), and PKR (eukaryotic translation initiation factor 2 alpha kinase 2, also known as EIF2AK2), which are stimulated in response to trascription malfunctions.

Exemplary transcription malfunctions which stimulate the ER stress response pathways include, but are not limited to accumulation of unfolded proteins, impaired protein glycosylation or disulfide bond formation, overexpression or mutations in proteins and accumulation of double stranded RNA.

Thus, the ER stress response pathways act towards halting protein translation, degrading misfolded proteins, and activating the signalling pathways that lead to increasing the production of molecular chaperones, lipids and other biological substances required to restore homeostasis.

According to some embodiments of the invention, the ER stress inhibitor is a naturally-occurring molecule selected from the group consisting of: Quercetin, Ursolic acid, curcumin, taurochenodeoxycholic acid, chenodeoxycholic acid, and oleanolic acid.

According to some embodiments of the invention, the ER stress inhibitor is a chemically-synthesized molecule selected from the group consisting of: KIRA6, 3HNA, MKC-3946, TUDCA, 4-PBA, Telmisartan, N-Acetyl cysteine, Tempol, and Olmesartan.

As described, the method according to some embodiments of the invention requires a combination of protective agents for reducing the renal toxicity that may be caused by the kidney damaging agent. The protective agents comprise:

a SGLT2 inhibitor; and

an agent selected from the group consisting of: a PPARA activator, a C/EBP inhibitor, a PPARG inhibitor, or an ER stress inhibitor.

According to some embodiments of the invention, the SGLT2 inhibitor is chemically-synthesized molecule selected from the group consisting of: Empagliflozin, Dapagliflozin, Canagliflozin, Ertugliflozin, Ipragliflozin, Luseogliflozin, Remogliflozin etabonate, Sotagliflozin and Tofogliflozin.

According to some embodiments of the invention, the activator of PPARA is a chemically-synthesized molecule.

According to some embodiments of the invention, the activator of PPARA is a naturally-occurring molecule.

A non-limiting example of a naturally-occurring PPARA activator (agonist) is 9CLA (cis-9,trans-11-octadecadienoic acid 18:2).

According to an aspect of some embodiments of the invention there is provided a composition comprising:

(i) a kidney damaging agent;

(ii) a PPARA activator; and

(iii) an inhibitor of a cellular pathway selected from the group consisting of C/EBP, PPARG, ER stress, GLUT2 and SGLT1/2.

According to an aspect of some embodiments of the invention there is provided a composition comprising:

(i) a kidney damaging agent;

(ii) a SGLT2 inhibitor; and

(iii) a PPARA activator, a C/EBP inhibitor, a PPARG inhibitor, or an ER stress inhibitor.

According to some embodiments of the invention, the composition further comprising a pharmaceutically acceptable carrier.

According to an aspect of some embodiments of the invention there is provided an article of manufacture, comprising:

(i) a kidney damaging agent;

(ii) a peroxisome proliferator-activated receptor alpha (PPARA) activator; and

(iii) an inhibitor of a cellular pathway selected from the group consisting of CCAAT/enhancer-binding protein (C/EBP), peroxisome proliferator-activated receptor gamma (PPARG), endoplasmic reticulum (ER) stress, glucose transporter 2 (GLUT2) and sodium-glucose cotransporter 1 and 2 (SGLT1/2).

According to some embodiments of the invention, wherein in the method, the article of manufacture or the composition at least the PPARA activator and the inhibitor in (iii) are in a co-formulation.

According to an aspect of some embodiments of the invention there is provided an article of manufacture, comprising:

(i) a kidney damaging agent;

(ii) a sodium-glucose linked transporter type 2 (SGLT2) inhibitor; and

(iii) a peroxisome proliferator-activated receptor alpha (PPARA) activator, a CCAAT/enhancer-binding protein (C/EBP) inhibitor, a peroxisome proliferator-activated receptor gamma (PPARG) inhibitor, or an endoplasmic reticulum (ER) stress inhibitor.

According to some embodiments of the invention, wherein in the method, the article of manufacture or the composition at least the SGLT2 inhibitor and the inhibitor in (iii) are in a co-formulation.

According to some embodiments of the invention, wherein in the method, the article of manufacture or the composition at least the SGLT2 inhibitor and the activator in (iii) are in a co-formulation.

According to some embodiments of the invention, the article of manufacture of some embodiments of the invention is for use in treating a disease for which the kidney damaging agent is therapeutic.

According to some embodiments of the invention, the article of manufacture further comprises appropriate instructions for use and labels indicating FDA approval for use in preventing and/or reducing renal toxicity.

It should be noted that the effect of the protective agents on the kidney (e.g., protecting against renal damage, and/or dyslipidemia and/or hyperglycemia) can be evaluated by testing the level of various acceptable markers in a blood and/or a urine sample of the subject. For example, using a blood sample one can detect the level of estimated glomerular filtration rate (eGFR), creatinine, lactate dehydrogenase (LDH), uric acid, sodium, potassium, calcium, phosphorus, alkaline phosphatase, and/or urea. Additionally or alternatively, using a urine sample, one can detect the level of PH, protein, blood and/or albumin.

The protective agents that are used by the methods, the compositions or the article of manufacture of some embodiments of the invention [e.g., the PPARA activator and the inhibitor of a cellular pathway selected from the group consisting of C/EBP, PPARG, ER stress, GLUT2 and SGLT1/2; and/or the SGLT2 inhibitor and an agent selected from the group consisting of a PPARA activator, a C/EBP inhibitor, a PPARG inhibitor, and an ER stress inhibitor] may be administered to the subject per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to at least one agent from the protective agents, e.g., the PPARA activator and the inhibitor of a cellular pathway selected from the group consisting of C/EBP, PPARG, ER stress, GLUT2 and SGLT1/2; and/or the SGLT2 inhibitor and an agent selected from the group consisting of a PPARA activator, a C/EBP inhibitor, a PPARG inhibitor, and an ER stress inhibitor.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (agent that decreases lipid accumulation) effective to prevent, alleviate or ameliorate symptoms of a disorder (e.g., cancer/anthrax infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue or blood levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

In the context of a combination therapy, the kidney-damaging agent may be administered by the same route of administration (e.g. intrapulmonary, oral, enteral, etc.) as of at least one of the protective agents [e.g., the PPARA activator and the inhibitor of a cellular pathway selected from the group consisting of C/EBP, PPARG, ER stress, GLUT2 and SGLT1/2; and/or the SGLT2 inhibitor and an agent selected from the group consisting of a PPARA activator, a C/EBP inhibitor, a PPARG inhibitor, and an ER stress inhibitor]. In the alternative, the kidney-damaging agent may be administered by a different route of administration to the protective agents [e.g., the PPARA activator and the inhibitor of a cellular pathway selected from the group consisting of C/EBP, PPARG, ER stress, GLUT2 and SGLT1/2; and/or the SGLT2 inhibitor and an agent selected from the group consisting of a PPARA activator, a C/EBP inhibitor, a PPARG inhibitor, and an ER stress inhibitor].

The kidney-damaging agent can be administered immediately prior to (or after) the protective agent, on the same day as, one day before (or after), one week before (or after), one month before (or after), or two months before (or after) the protective agent, and the like.

The kidney-damaging agent and the protective agents (e.g. e.g., the PPARA activator and the inhibitor of a cellular pathway selected from the group consisting of C/EBP, PPARG, ER stress, GLUT2 and SGLT1/2; and/or the SGLT2 inhibitor and an agent selected from the group consisting of a PPARA activator, a C/EBP inhibitor, a PPARG inhibitor, and an ER stress inhibitor) can be administered concomitantly, that is, where the administering for each of these reagents can occur at time intervals that partially or fully overlap each other. They may be administered in a single formulation or in separate formulations. The kidney-damaging agent and the protective agents can be administered during time intervals that do not overlap each other. For example, the kidney-damaging agent can be administered within the time frame of t=0 to 1 hours, while the protective agents can be administered within the time frame of t=1 to 2 hours. Also, the kidney-damaging agent can be administered within the time frame of t=0 to 1 hours, while the protective agents can be administered somewhere within the time frame of t=2-3 hours, t=3-4 hours, t=4-5 hours, t=5-6 hours, t=6-7 hours, t=7-8 hours, t=8-9 hours, t=9-10 hours, and the like. Moreover, the protective agents can be administered somewhere in the time frame of t=minus 2-3 hours, t=minus 3-4 hours, t=minus 4-5 hours, t=5-6 minus hours, t=minus 6-7 hours, t=minus 7-8 hours, t=minus 8-9 hours, t=minus 9-10 hours.

It will be appreciated that the protective agents may be formulated in a single composition together with the nephrotoxic agent.

Thus, the present invention contemplates compositions (e.g. pharmaceutical compositions) comprising a single acceptable carrier and, as active agents:

(i) a kidney-damaging therapeutic agent;

(ii) a PPARA activator; and

(iii) an inhibitor of a cellular pathway selected from the group consisting of C/EBP, PPARG, ER stress, GLUT2 and SGLT1/2.

Additionally or alternatively the present invention contemplates compositions (e.g., pharmaceutical compositions) comprising a single acceptable carrier and, as active agents:

(i) a kidney damaging agent;

(ii) a SGLT2 inhibitor; and

(iii) a PPARA activator, a C/EBP inhibitor, a PPARG inhibitor, or an ER stress inhibitor.

The protective agents of the present invention and the kidney-damaging agent are typically provided in combined amounts to achieve therapeutic and/or prophylactic effectiveness. This amount will evidently depend upon the particular compound selected for use, the nature and number of the other treatment modality, the condition(s) to be treated, prevented and/or palliated, the species, age, sex, weight, health and prognosis of the subject, the mode of administration, effectiveness of targeting, residence time, mode of clearance, type and severity of side effects of the composition and upon many other factors which will be evident to those of skill in the art. The kidney-damaging agent will be used at a level at which a therapeutic or prophylactic effect in combination with the protective agents is observed.

The kidney-damaging agent may be administered (together with the protective agents) at a gold standard dosing as a single agent, below a gold standard dosing as a single agent or above a gold standard dosing as a single agent.

According to specific embodiments, the kidney-damaging agent is administered above the gold standard dosing as a single agent.

As used herein the term “gold standard dosing” refers to the dosing which is recommended by a regulatory agency (e.g., FDA), for a given tumor at a given stage.

According to specific embodiments, the kidney-damaging agent is administered using a regimen which is different to the gold standard regimen when used as a single agent (e.g. it may be provided for a longer length of time).

According to other specific embodiments, the kidney-damaging agent is administered (in combination with the protective agents) at a dose that is associated with kidney damage when used as a single agent.

Thus, in one embodiment, the amount of the kidney-damaging agent (when used in combination therapy) is above the minimum dose used for therapeutic or prophylactic effectiveness when used as a single therapy (e.g. 110%, or 125% to 175% of the minimum dose). The therapy is rendered more effective because higher doses of the active agent can be used to treat the disease whilst the protective agents decrease the negative side-effect of renal toxicity, the combinations are effective overall.

In an alternative embodiment, the kidney-damaging agent of the present invention and the protective agents are synergistic with respect to their side effects. That is to say that the side-effects caused by the protective agents in combination with the kidney-damaging agent are less than would be anticipated when the equivalent therapeutic effect is provided by either the kidney-damaging agent or each of the protective agents when used separately.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It should be noted that detection of renal toxicity can be performed in vitro using renal cells, or organoids comprising same.

The present inventors have previously disclosed a microphysiological kidney-on-chip platform for testing the effects of drugs on kidney cells (PCT IL2020/050173). The microphysiological kidney-on-chip platform comprises structurally and functionally mature proximal tubular organoids, which show multiple longitudinal polarized tubules with evidence of apical brush borders. These three-dimensional organoids, made from Human Kidney-2 cells (HK-2), primary Renal Proximal Tubule Epithelial Cells (RPTEC) or Upcyte Proximal Tubule Cells (UPC PTC) are vascularized and may be embedded with microsensors for real-time oxygen measurement in the tissue. The organoids are then placed in a multi-well perfused bioreactor. These bioreactors are microfluidically linked to electrochemical sensors allowing for continuous measurements of the main players of central carbon metabolism (including glucose, lactate, glutamine and glutamate) from the outflow. This level of information contributes to the accuracy of metabolic flux analysis of the organoids, providing a window into the dynamic changes of their metabolism whilst in the presence of potentially nephrotoxic agents.

The organoids comprising mature, polarized kidney epithelial cells and endothelial cells, and three-dimensional longitudinal tubules having at least two openings, each organoid having at least one central lumen, wherein less than 50% of the cells of the organoid express a fetal marker.

As used herein the term “organoid” refers to an artificial three-dimensional aggregate of live cells of at least two cell types. The organoid of this aspect of the present invention is generated in-vitro as further described herein below.

The organoids are typically between 100-2000 μm in diameter (for example between 200-1000 μm in diameter and may comprise between about 500-100,000 cells (for example between 1,000 and 75,000 cells).

The organoid(s) may comprise only human cells, and/or non-human cells.

The organoid(s) may comprise at least one epithelial lined tubule (each tubule having at least two openings). The tubules are surrounded by endothelial vessels.

The organoid can carry out at least one function of a kidney, for example glucose reabsorption.

The organoids may comprise at least one central lumen, at least two central lumens, at least three central lumens or more.

The organoid can be typically generated from mature human renal cells and as such does not express fetal markers.

The organoid may comprise mature polarized human kidney cells.

Preferably, the polarized cells of the organoid comprise epithelial cells. For example the apical surface of epithelial cells which face the tubular lumens are different in protein and lipid composition to the basolateral surface of the cells.

Preferably, less than 50% of the cells of the organoid express a fetal marker, less than 40% of the cells of the organoid express a fetal marker, less than 30% of the cells of the organoid express a fetal marker, less than 20% of the cells of the organoid express a fetal marker, less than 10% of the cells of the organoid express a fetal marker, as measured by immunohistochemistry and/or RT-PCR. In one embodiment, none of the cells of the organoid express a fetal marker. In yet another embodiment, the cells of the instant organoid express at least 10% less, 20% less, at least 30% less, at least 40% less or even at least 50% less fetal marker than a kidney organoid which is generated from non-mature cells such as embryonic stem cells, as measured under identical conditions by immunohistochemistry and/or RT-PCR.

An example of a fetal marker which is not expressed in the organoids disclosed herein is KSP (CDH16; Cadherin-16). Additional examples include OSR1 (Protein odd-skipped-related 1), WT1 (Wilms tumor 1), GDNF (Glial cell line-derived neurotrophic factor), CITED1 (Cbp/p300-interacting transactivator 1), HOXD11 (Homeobox protein Hox-D 11), Wnt4 (wingless-type MMTV integration site family, member 4), Lhx1 (Liml; LIM homeobox protein 1), Nr2f2 (COUP-TFII; nuclear receptor subfamily 2, group F, member 2) and MUCi (mucin 1, cell surface associated).

Preferably, the organoid used to detect renal toxicity is not generated from pluripotent stem cells (e.g. embryonic stem cells) and/or renal progenitor cells.

Exemplary cells which may be used to generate the organoid disclosed herein include, but are not limited to Human Kidney-2 cells (HK-2), primary Renal Proximal Tubule Epithelial Cells (RPTEC), Human embryonic kidney 293 (HEK293) and primary kidney podocyte cells (NhKP).

The organoid used to detect renal toxicity may typically express markers of mature cells, as measured by immunohistochemistry and/or RT-PCR. Exemplary markers expressed by the organoid include, but are not limited to ALPI (alkaline phosphatase, intestinal), Aqp1 (aquaporin 1), Cldn10 (claudin 10), Cldn11 (Osp; claudin 11), Cldn2 (claudin 2), DPP4 (DPPIV; dipeptidyl peptidase 4), Enpep (Aminopeptidase A; glutamyl aminopeptidase), GGCT (gamma-glutamylcyclotransferase), LAP3 (LAP; leucine aminopeptidase 3), MME (CD10; membrane metalloendopeptidas), Slc36a2 (solute carrier family 36 [proton/amino acid symporter], member 2), SLC5A1 (Na/Gluc1; solute carrier family 5 member 1), Slc6a18 (solute carrier family 6 [neurotransmitter transporter], member 18), Slc6a19 (solute carrier family 6 [neurotransmitter transporter], member 19), Slc6a20a (solute carrier family 6 [neurotransmitter transporter], member 20A), Slc6a20b (solute carrier family 6 [neurotransmitter transporter], member 20B).

The organoid used to detect renal toxicity may express at least one, two, three, four, five, six, seven, eight, nine or more of the above mentioned markers.

The diameter of a tubules comprised in the organoid used to detect renal toxicity is typically between about 10 to 200 microns (e.g. between 50-100 microns in diameter).

The length of the tubules comprised in the organoid used to detect renal toxicity is typically between 100-1000 microns (e.g. between 200-600 microns).

The organoid used to detect renal toxicity may be vascularized or non-vascularized.

As used herein, the term “vascularizes organoid” refers to formation of at least a part of a 3D blood vessel network around the organoid. Typically, the blood vessel network is comprised of endothelial cells. The vasculature may be at any stage of formation as long as it comprises at least one 3D endothelial structure. Examples of 3D endothelial structures include, but are not limited to tube-like structures, preferable those comprising a lumen.

The organoids used to detect renal toxicity may be embedded with at least one microsensor for oxygen monitoring (e.g. real-time oxygen monitoring).

The microsensors are typically capable of measuring oxygen uptake (or consumption) of the cells.

In one embodiment, the microsensors are lifetime-based luminescence-quenching (LBLQ) microparticles or nanoparticles. The microparticles or nanoparticles are optionally and preferably used for measuring oxygen by determining their phase modulation. The advantage of using microparticles or nanoparticles as an oxygen sensor is that such oxygen measurement can be done without calibrating the number of cells and there is no need to operate in tiny volumes.

Microparticles and nanoparticles useful as oxygen sensors suitable for the present embodiments are found in U.S. Published Application No. 20150268224, published on Sep. 24, 2015, the contents of which are hereby incorporated by reference.

In one embodiment, the microsensor is a nanoparticle or microparticle (e.g. about 50 microns in diameter) loaded with a ruthenium-based dye.

The ratio of microsensor to cells comprised in the cell suspension which is used to generate the organoid is typically between 0.5-4 mg (milligram) per milliliter of cell suspension.

Other exemplary microsensors which can be used to measure oxygen uptake include but are not limited to 50 micrometer-diameter polystyrene microbeads loaded with ruthenium-phenanthroline-based phosphorescence dye such as CPOx-50-RuP (Colibri Photonics) or 200 nm-diameter beads OXNANO (Pyro Science).

To generate the organoids used to detect renal toxicity renal cells such as Human Kidney-2 cells (HK-2) or primary Renal Proximal Tubule Epithelial Cells (RPTEC) are cultured at a density of about 0.5-10×10⁴ (e.g. 7.5×10⁴ cells) per 1.5 mm well. Preferably, the cells are cultured on an extracellular matrix (e.g., Matrigel™ or laminin) in the presence of a culture medium. The cells are cultured under conditions that promote organoid formation (e.g. at 37° C. in a humidified incubator with about 5% CO₂ for 10-24 hours).

According to a particular embodiment, the organoids used to detect renal toxicity are cultured in a bioreactor which is continuously perfused with cell culture medium (for example a microfluidic array). The microfluidic array may comprise a plurality of wells for culturing the organoids.

A perfusion element may be used for generating, in a controlled manner, a flow of a perfusion medium onto the array, wherein the flow is controlled so as to ensure continuous perfusion. During the flow, signals indicative of one or more physiological parameters may be collected from the array. The signals can be recorded on a computer readable medium, preferably a non-transitory computer readable medium. Alternatively or additionally, the signals can be analyzed to determine one or more physiological parameters that are characteristic of the cells of the organoid on the array.

The organoids used to detect renal toxicity can be placed in a multi-well plate comprising an array of wells each containing a distinct organoid therein, wherein a size of each organoid is within less than 20% or less than 15% or less than 10% from an average size of all organoids occupying the array.

The organoids used to detect renal toxicity which are present in the wells of the multi-well plate can be homogenous in terms of size and/or cell number. For example, each well may have a single (distinct organoid), wherein all organoids are homogenous.

When the organoid is cultured in a microfluidic array, the microwell may comprise an insert so as to protect the cells from the negative effects of shear force. The insert may be fabricated from materials known in the art to be compatible with tissue culturing—for example polydimethylsiloxane (PDMS), glass, Poly(methyl methacrylate) (PMMA), Cyclic olefin copolymer (COC), Polycarbonate, or Polystyrene The array may further comprise a temperature sensor, a glucose sensor and/or a lactate sensor and/or a glutamine sensor.

The lactate and/or glucose sensor may be electrochemical—e.g. allowing for amperometric measurement of lactate and/or glucose.

In one embodiment pH of the medium is measured as a surrogate for lactate measurement.

The array of comprising the organoids may further comprise at least one of: an electronic control circuit for signal modulation and read-out, a light source (e.g., LED) for excitation (e.g., of the oxygen sensing particles), an optical filter set (e.g., 531/40, 555, 607/70 nm) and a detector unit containing a photomultiplier (PMT). One skilled in the art would appreciate that the sensing particles can be excited by various wave lengths depending on the specific sensing particles used. Accordingly, emission may be read at various wave lengths as well.

Preferably the array has a three-electrode design in which the counter and reference electrodes are separated. The reference electrode is used to measure the working electrode potential without passing current through it, while the counter electrode closes a circuit, allowing current to pass.

Thus, determining the toxic effect of the protective agents on the kidney can be performed in a method comprising:

(i) providing an organoid which comprises at least one microsensor for oxygen monitoring;

(ii) culturing the organoid under physiological conditions in the presence of the candidate agent; and

(iii) performing real-time measurements of oxygen consumption of the organoid, wherein a decrease in oxygen consumption of the organoid in the presence of the candidate agent as compared to the oxygen consumption of the organoid in the absence of the candidate agent is indicative that the candidate agent has a toxic effect on the kidney.

Examples of agents that may be tested include, but are not limited to therapeutic agents, diagnostic agents, imaging agents such (e.g. dyes), food, cosmetics, and agents suspected of having environmental nephrotoxic effect.

Exemplary measurements of oxygen consumption that can be carried out on the organoids include at least one of the following:

a. determination of the percentage of the reduction of the oxygen consumption caused by the agent;

b. determination of the duration of the exposure to the candidate agent that causes a reduction (for example a 10% reduction, a 20% reduction, a 30% reduction, a 40% reduction, a 50% reduction etc. in the oxygen consumption; and

c. determination of the concentration of the candidate agent that causes a 10% reduction, a 20% reduction, a 30% reduction, a 40% reduction, a 50% reduction of the oxygen consumption.

In a particular embodiment wherein the oxygen sensing particle is a ruthenium-phenanthroline-based particle, the particles are excited by 532 nm and a 605 nm emission is read, so as to measure phosphorescence decay, substantially in real time.

The agents which are being tested are preferably present at a concentration that causes less than 50%, 40%, 30%, 20%, 10% of the cells in the organoid to die in 24 hours.

As well as measuring the amount of oxygen in the system outflow (so as to determine oxygen consumption of the cells), the present inventors envisage measuring lactate in the system outflow (so as to determine lactate production of the cells) and/or measuring glucose in the system outflow (so as to determine glucose usage of the cells) and/or measuring glutamine in the system outflow (so as to determine glutamine production of the cells).

Glutamine, like glucose and lactate, can also be measured electrochemically. For example, glutaminase and glutamate oxidase enzymes may be immobilized in a membrane. Glutamine is transformed to glutamic acid by glutaminase, and the glutamic acid is transformed by glutamate oxidase to form a detectable reaction product using amperometric or potentiometric sensor. Sensors can be purchased from Innovative Sensor Technologies (Las Vegas, Nev.). Other methods of measuring glutamine production are described in WO1988010424 A1, U.S. Pat. No. 4,780,191.

Usually, glutaminase and glutamate oxidase enzymes are immobilized in a membrane. Glutamine is transformed to glutamic acid by glutaminase, and the glutamic acid is transformed by glutamate oxidase to form a detectable reaction product using amperometric or potentiometric sensor. Sensors can be purchased from Innovative Sensor Technologies (Las Vegas, Nev.).

According to a particular embodiment, the constituents of the perfusion medium are such that at least one metabolic pathway of the organoid is eliminated. Thus for example the perfusion medium may be deficient in at least one nutrient type.

It will be appreciated that the term “deficient” does not necessarily mean that the medium is totally devoid of that constituent, but that it may be present in limited amounts such that the at least one metabolic pathway of the cells of the organoid is eliminated. Thus, for example trace amounts of the constituent may be present in the proliferation medium.

Elimination of a pathway (i.e. bypassing of a pathway, or shunting away from the use of the pathway) also does not have to be total. In one embodiment, utilization of that pathway is at least 10 times, 20 times, 50 times or even 100 times lower than an alternate pathway which generates the same end-product and/or which uses the same starting material.

It will be appreciated that the term “elimination of a pathway” may refer to the bypassing of, or shunting away from, only one or both directions of a pathway.

Elimination of a pathway is important such that when metabolic flux is calculated this pathway (or pathway in one particular direction) can be neglected.

The eliminated pathway includes for example the lipid oxidation pathway, glycolysis, glutaminolysis, urea cycle, lipogenesis, cholesterol synthesis, mevalonate pathway, and the anaplerotic reactions replenishing the TCA cycle (e.g. valine, isoleucine).

Thus for example in the case where the medium is lipid deficient or limited in lipids, the amount of lipids in the medium is such that fatty acid uptake is at least 10 fold, 20 fold, 50 fold or even 100 fold lower than glucose uptake in the cell.

Examples of nutrient types which may be deficient include, but are not limited to a fatty acid, triglyceride, cholesterol, monosaccharide, pyruvate, glycerol and an amino acid.

For example, if pyruvate and glycerol are deficient in the perfusion medium, the metabolic pathways into glycolysis are eliminated. This is useful for determining the amount of glycolysis of the cell.

For example if lipids such as fatty acids and triglycerides are deficient in the perfusion medium, the lipid oxidation pathway is eliminated. This is useful for determining the amount of glycolysis, oxidative phosphorylation, and/or glutaminolysis of the cells.

For example, the constituents of the perfusion medium can be selected which induce or inhibit glycolytic flux. Then, measurements of oxygen, glucose and/or lactate can be made as further detailed hereinabove and used for determining glycolytic capacity, glycolytic reserve and/or non-glycolytic acidification. In a representative example, which is not to be considered as limiting, non-glycolytic acidification is determined before perfusion of the environment altering constituents, glycolytic capacity can be determined following perfusion with saturating concentration of glucose, which is used by the cells through the glycolytic pathway to produce ATP, NADH, water, and protons, and glycolytic reserve is determined following perfusion with glycolysis inhibitor, such as, but not limited to, 2-deoxyglucose, which inhibits glycolysis by binding to hexokinase.

Exemplary metabolic flux (e.g. the rate of turnover of molecules through a particular metabolic pathway), that may be calculated using the system described herein are set forth in Table 3 herein below.

TABLE 3
Table 3.
Metabolic flux

Glucose + Pᵢ ↔ Glucose 6-P + H₂0
Glucose 6-P ↔ Fructose 6-P
Fructose 6-P + Pi ↔ Fructose 1,6-P₂ + H₂0
Fructose 1,6-P₂ ↔ 2 Glyceraldehyde 3-P
Glyceraldehyde 3-P + NAD⁺ + Pi+ ADP ↔
Phosphoenolpyruvate + NADH + H⁺ + ATP
Phosphoenolpyruvate + ADP → Pyruvate + ATP
Pyruvate + CoA + NAD⁺ → Acetyl-CoA + CO₂ + NADH
Lactate + NAD⁺ ↔ Pyruvate + NADH + H⁺
Acetyl-CoA + Oxaloacetate + H₂O→ Citrate + CoA + H⁺
Citrate + NAD+ ↔ 2-oxo-gluterate + NADH + CO₂
2-oxo-gluterate + NAD⁺ + CoA → Succinyl-CoA + NADH + CO₂ + H⁺
Succinyl-CoA + Pᵢ + GDH + FDH ↔ Fumarate + GTP + FADH₂ + CoA
Fumarate + H₂O ↔ Malate
Malate + NAD+ ↔ Oxaloacetate + NADH + H⁺
Ornithine + CO₂ + NH₄⁺ + 2 ATP + H₂O ↔ Citrulline + 2 ATP + 2Pᵢ + 3H⁺
Citrulline + Aspartate + ATP → Arginine + Fumarate + AMP + PPᵢ
Arginine uptake
Ammonia production
Ornithine Output
Alanine + 0.5 NAD⁺ + 0.05 NADP⁺ + H₂O →
Pyruvate + NH₄⁺ + 0.5 NADH + 0.5 NADPH + H⁺
Alanine Uptake
Serine → Pyruvate + NH₄⁺
Serine Uptake
Cysteine + 0.5 NAD⁺ + 0.5 NADP⁺ + H₂O + SO₃ ²⁻ →
Pyruvate + Thiosulfate + NH₄⁺ + 0.5 NADH + 0.5 NADPH + H⁺
Cysteine Uptake
Threonine + NAD⁺ → Glycine + Acetyl-CoA + NADH
Glycine + NAD⁺ + H₄folate ↔ N⁵,N¹⁰-CH₂H₄folate + NADH + CO₂ + NH₄⁺ + H⁺
Glycine Uptake
Tryptophan + 3 H₂O + 3 O₂ + CoA + 3 NAD⁺ + FAD →
3CO₂ + FADH₂ + 3NADH + 4H⁺ + NH₄⁺ + Acetoacetatyl-CoA
Propionyl-CoA + CO₂ + ATP → Succinyl-CoA + AMP + PPᵢ
Lysine + 3H₂O + 5 NAD⁺ + FAD + CoA →
2 NH₄⁺ + 5 NADH + 5 H⁺ + FADH₂ + 2 CO₂ + Acetoacetatyl-CoA
Phenylalanine + H₄biopterin + O₂ → Tyrosine + H₂biopterin + H₂O
Tyrosine + 0.5 NAD⁺ + 0.5 NADP⁺ + H₂O + 2 O₂ →
NH₄⁺ + CO₂ + 0.5 NADH + 0.5 NADPH + H⁺ + Fumarate + Acetoacetate
Tyrosine Uptake
Glutamate + 0.5 NAD⁺ + 0.5 NADP⁺ + H₂O ↔
NH₄⁺ + 2-oxo-gluterate + 0.5 NADH + 0.5 NADPH + H⁺
Glutamate Uptake
Glutamine + H₂O → Glutamate + NH₄⁺
Ornithine + NAD⁺ + NADP⁺ + H₂O Glutamate + NH₄⁺ + NADH + NADPH + H⁺
Proline + 0.5 O₂ + 0.5 NAD⁺ + 0.5 NADP⁺ →
Glutamate + 0.5 NADH + 0.5 NADPH + H⁺
Histidine + H₄folate + 2H₂O → NH₄⁺ + N5-formiminoH₄folate + Glutamate
Methionine + ATP + Serine + NAD⁺ + CoA →
PPᵢ + Pᵢ + Adensosine + Cysteine + NADH + Propionyl-CoA + CO₂ + NH₄⁺
Aspartate + 0.5 NAD⁺ + 0.5 NADP⁺ + H₂O ↔
Oxaloacetate + NH₄⁺ + 0.5 NADH + 0.5 NADPH + H⁺
Aspartate Uptake
Asparagine + H₂O → Aspartate + NH₄⁺
8 Acetyl-CoA + 7 ATP + 14 NADPH + 14 H⁺ →
Palmitate + 8 CoA + 6 H₂O + 7 ADP + 7Pᵢ + 14 NADP⁺
2 Acetyl-CoA ↔ Acetoacetyl-CoA + CoA
Acetoacetyl-CoA + H₂O → Acetoacetate + CoA
Acetoacetate Output
Acetoacetate + NADH + H⁺ ↔ β-hydroxybutyrate + NAD⁺
NADH + H⁺ + 0.5 O₂ + 3 ADP → NAD⁺ + H₂O + 3 ATP
FADH₂ + 0.5 O₂ + 2 ADP → FAD + H₂O + 2 ATP
O₂ Uptake
Glucose 6-P + 12 NADP⁺ + 7 H₂O → 6 CO₂ + 12 NADPH + 12 H⁺ + Pᵢ
Valine + 0.5 NADP⁺ + CoA + 2 H₂O + 3.5 NAD⁺ + FAD →
NH₄⁺ + Propionyl-CoA + 3.5 NADH + 0.5 NADPH + 3 H⁺ + FADH₂ + 2 CO₂
Isoleucine + 0.5 NADP⁺ + H₂O + 2.5 NAD⁺ + FAD + 2 CoA →
NH₄⁺ + Propionyl-CoA + Acetyl-CoA + 2.5 NADH + 0.5 NADPH + 3H⁺ +
FADH² + CO₂
Leucine + 0.5 NADP⁺ + H₂O + 1.5 NAD⁺ + FAD + ATP + CoA →
NH₄⁺ + 1.5 NADH + 0.5 NADPH + 2 H⁺ + FADH₂ + ADP + Pᵢ +
Acetoacetate + Acetyl-CoA
Threonine uptake
Lysine Uptake
Phenylalanine Uptake
Glutamine Uptake
Proline Uptake
Histidine Uptake
Methionine Uptake
Asparagine Uptake
Valine Uptake
Isoleucine Uptake
Leucine Uptake
Protein Synthesis
Triglyceride ↔ Glycerol + 3 Palmitate
Triglyceride Uptake
Glycerol Uptake
Palmitate Uptake
Glucose-6-P + UTP + H₂O ↔ Glycogen + 2 Pᵢ + UDP
Glycerol + NAD⁺ ↔ Glyceraldehyde 3-P + NADH + H⁺
18 Acetyl-CoA + 25 NADPH + NADH + 26 H⁺ + 18 ATP + 11 O₂ →
Cholesterol + 25 NADP⁺ + NAD⁺ + 18 ADP + 6 Pᵢ + PPᵢ + 8 CO₂ +
6 H₂O + 18 CoA + HCOOH
Cholesterol + 5 NADPH + H⁺ + 3 O₂ + ATP + 2 CoA + FAD ↔
Choloyl-CoA + 5 NADP⁺ + 2 H₂O + ADP + PPᵢ + FADH₂ + Propionil-CoA
Cholesterol Output
CO₂ output

It is expected that during the life of a patent maturing from this application many relevant useful agent that have the negative side-effect of being kidney damaging will be developed and the scope of the term kidney damaging agent is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Testing the effect of combination therapy on human kidney cells exposed to kidney damaging agents—Human primary proximal tubule cells (hPTC) were seeded at density of 10,000 cells/cm² in a black 96-well plate. When the cells reach 80% density, treatments were induced. The kidney damaging agents Cyclosporine A, Cisplatin and Gentamicin were diluted in hPTC medium to reach a final concentration of 1 μM, 1 μM and 1 mM respectively. The drugs were induced either alone (controls) or in combination with Empagliflozin (SGLT2i) at 5 μM or Fenofibrate at 20 μM, themselves in combination with Quercetin at 20 μM. All different solutions included 1× Red Phospholipidosis staining reagent as well. hPTC were incubated with the different combinations for 48 hours at 37° C. 5% CO₂ 95% O₂. After 48 hours, the media were removed and replaced with Green LipidTox stain to stain for neutral lipids in green (diluted at 1:200 in hPTC medium). After 45 minutes of incubation at 37° C., the plate was mounted onto a Zeiss epifluorescent microscope for quantification. Each condition was tested in three biological replicates, and light settings were the same for all the images. Images were analyzed with ImageJ.

hPTC medium—hPTC culture medium is composed of MCDB 153 basal medium (Sigma-Aldrich, USA) supplemented with 0.5% fetal bovine serum (BI, Israel), Insulin, transferrin and selenium (ITS, Gibco, USA), 0.1 μM dexamethasone (Sigma-Aldrich, USA), 10 ng ml⁻¹ Epithelial Growth Factor (EGF, Peprotech, USA), 5 μM Triiodothyronine (T3, Sigma, USA), 0.5 μg ml⁻¹ Epinephrin (Sigma. USA), 100 U ml⁻¹ penicillin, and 100 μg ml⁻¹ streptomycin (BI, Israel).

### Example 1

### Combination Therapy which Prevents Kidney Damage and Dyslipidemia

Experimental Results

Cyclosporine A, Cisplatin and Gentamicin induced lipotoxicity of the kidneys can be prevented by co-administration of the drugs with a SGLT2 inhibitor like empagliflozin (FIGS. 1C, 2C and 3C). The addition of quercetin together with empagliflozin reduced phospholipidosis by 5-folds more (FIGS. 1E, 2E and 3E), showing increased safety.

Cyclosporine A, Cisplatin and Gentamicin induced lipotoxicity of the kidneys can also be prevented by co-administration of the drugs with a PPARAα agonist like fenofibrate (FIGS. 1B, 2B and 3B). The addition of quercetin together with fenofibrate reduced phospholipidosis by 10-folds more, (FIGS. 1D, 2D and 3D) showing increased safety.

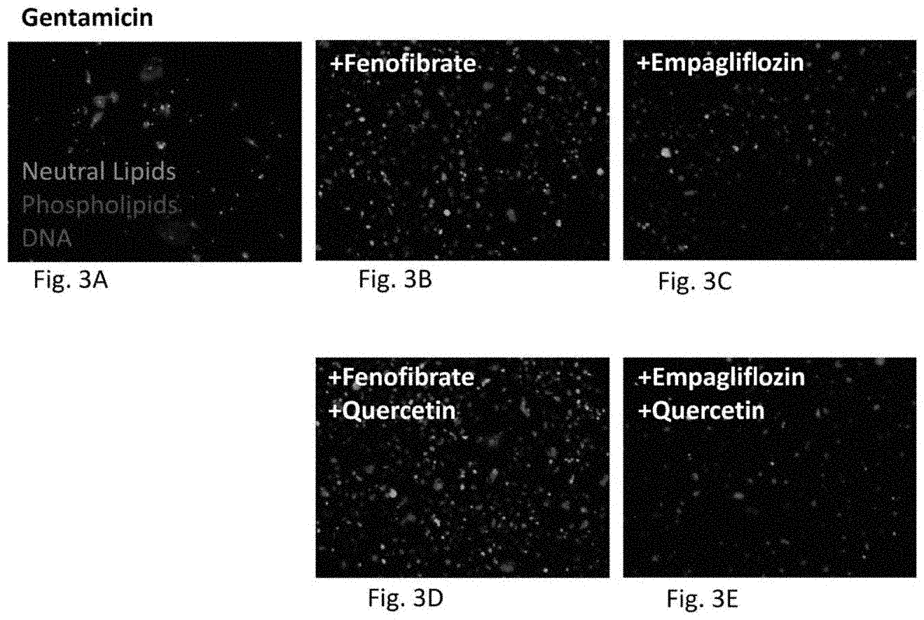

It is important to note that 1 mM gentamicin induced massive cell death by itself (FIG. 3A). However, co-administration of gentamicin with empagliflozin or fenofibrate dramatically increased the survival of human proximal tubule cells (hPTCs) (FIGS. 3B and 3C).

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for reducing renal tissue toxicity in a subject caused by a kidney damaging agent, the method comprising administering to the subject:
(i) a kidney damaging agent;
(ii) a peroxisome proliferator-activated receptor alpha (PPARA) activator; and
(iii) an inhibitor of a cellular pathway selected from the group consisting of CCAAT/enhancer-binding protein (C/EBP), peroxisome proliferator-activated receptor gamma (PPARG), endoplasmic reticulum (ER) stress, glucose transporter 2 (GLUT2) and sodium-glucose cotransporter 1 and 2 (SGLT1/2).

2. A method for reducing renal tissue toxicity in a subject caused by a kidney damaging agent, the method comprising administering to the subject:
(i) a kidney damaging agent;
(ii) a sodium-glucose linked transporter type 2 (SGLT2) inhibitor; and
(iii) a peroxisome proliferator-activated receptor alpha (PPARA) activator, a CCAAT/enhancer-binding protein (C/EBP) inhibitor, a peroxisome proliferator-activated receptor gamma (PPARG) inhibitor, or an endoplasmic reticulum (ER) stress inhibitor.

3. The method of claim 1, wherein said kidney damaging agent further causes dyslipidemia and/or hyperglycemia.

4. The method of claim 2, wherein said inhibitor or activator in (iii) is a naturally-occurring molecule.

5. The method of claim 2, wherein said inhibitor or activator in (iii) is a chemically-synthesized molecule.

6. The method of claim 1, with the proviso that when said kidney damaging agent is glufosamide then said inhibitor is not a sodium-glucose transport protein 2 (SGLT2) inhibitor.

7. The method of claim 2, with the proviso that said kidney damaging agent is not glufosamide.

8. The method of claim 1, wherein said PPARA activator in (ii) is selected from the group consisting of Fenofibrate, Benzofibrate, Ciprofibrate, Gemfibrozil, and Clofibrate.

9. The method of claim 2, wherein said SGLT2 inhibitor in (ii) is selected from the group consisting of: Empagliflozin, Dapagliflozin, Canagliflozin, Ertugliflozin, Ipragliflozin, Luseogliflozin, Remogliflozin etabonate, Sotagliflozin and Tofogliflozin.

10. The method of claim 2, wherein said PPARA activator in (iii) is 9CLA.

11. The method of claim 1, wherein said C/EBP inhibitor is Genistein.

12. The method of claim 1, wherein said PPARG inhibitor is Luteolin.

13. The method of claim 1, wherein said ER stress inhibitor Quercetin.

14. The method of claim 1, wherein said GLUT2 inhibitor is Phloretin.

15. The method of claim 1, wherein said SGLT1/2 inhibitor is Phlorizin.

16. The method of claim 1, wherein the subject has cancer and the kidney damaging agent is a therapeutic agent used to treat the cancer.

17. The method of claim 1, wherein the subject has undergone an organ or tissue transplant and the kidney damaging agent is an immunosuppressive agent.

18. The method of claim 1, wherein the subject does not have a metabolic disease.

19. The method of claim 1, wherein the kidney damaging agent is selected from the group consisting of an NSAID, an ACE Inhibitor, an angiotensin II Receptor Blocker, an aminoglycoside antibiotic, a radiocontrast dye, cyclosporine A (CsA) and a chemotherapeutic agent.

20. The method of claim 1, wherein the kidney damaging agent is selected from the group consisting of cisplatin, gentamicin and Cyclosporine A.
